(19) United States
(12) Patent Application Publication
(10) Pub. No.: US 2026/0112472 A1
(43) Pub. Date: Apr. 23, 2026

(54) METHOD AND SYSTEM FOR GENERATING ELECTRICAL STIMULATION PRESCRIPTION

(71) Applicant: GOLDEN SMART HOME TECHNOLOGY CORP., Hsinchu City (TW)

(72) Inventors: Fang-Cheng Liu, Hsinchu City (TW); Chuan-Feng Lin, Hsinchu County (TW); Tung-Shu YANG, Miaoli County (TW)

(21) Appl. No.: 19/362,742

(22) Filed: Oct. 20, 2025

(30) Foreign Application Priority Data
Oct. 20, 2024 (TW) 113139864

(51) Int. Cl. G16H 20/30; G16H 40/63

(52) U.S. Cl. CPC G16H 20/30; G16H 40/63

(57) ABSTRACT

Provided are a method and a system for generating an electrical stimulation prescription, including: obtaining, by a generative artificial intelligence module, an exclusive electrical stimulation prescription designed for a target patient and the target patient's feedback from executing the exclusive electrical stimulation prescription; adjusting, by the generative artificial intelligence module, the exclusive electrical stimulation prescription based on the feedback and at least one reference electrical stimulation prescription retrieved from a plurality of electrical stimulation prescriptions in a prescription database, so as to generate a new exclusive electrical stimulation prescription; displaying, by a prescription editing module, the new exclusive electrical stimulation prescription on a doctor's operating terminal for confirmation and/or editing; and receiving, by the prescription editing module, confirmation information of the new exclusive electrical stimulation prescription from the doctor's operating terminal and providing the new exclusive electrical stimulation prescription to the target patient for performing an electrical stimulation treatment.

### BACKGROUND

### Technical Field

The present disclosure relates to the technology for generating electrical stimulation prescriptions, and more particularly to methods and systems for generating electrical stimulation prescriptions that incorporate generative artificial intelligence technology.

### Description of Related Art

Electrical stimulation primarily utilizes physical electrical signals that are introduced into the body through a patient's skin to stimulate the patient's affected areas, thereby producing a therapeutic effect on the muscles or nerves of the affected areas.

Conventionally, patients, who require electrical stimulation, need to visit a medical institution in person to undergo an electrical stimulation treatment, while doctors must issue electrical stimulation prescriptions suitable for the patient's current condition based on their experience. If the patient experiences discomfort during the electrical stimulation treatment or if the doctor determines that the treatment effect is suboptimal, the doctor has to likewise adjust the existing electrical stimulation prescription based on their experience. Accordingly, timely issuing or adjusting an electrical stimulation prescription suitable for the patient's current condition becomes a significant burden for the doctor.

Therefore, developing a method and system for generating an electrical stimulation prescription that effectively addresses the aforementioned issues remains a problem that urgently needs to be solved.

### SUMMARY

The present disclosure provides a method for generating an electrical stimulation prescription that incorporates generative artificial intelligence technology to assist a doctor in issuing or adjusting electrical stimulation prescriptions.

In at least one embodiment, the method for generating an electrical stimulation prescription of the present disclosure comprises: obtaining, by a generative artificial intelligence module, an exclusive electrical stimulation prescription designed for a target patient; obtaining, by the generative artificial intelligence module, feedback from the target patient executing the exclusive electrical stimulation prescription; adjusting, by the generative artificial intelligence module, the exclusive electrical stimulation prescription based on the feedback and at least one reference electrical stimulation prescription retrieved from a plurality of electrical stimulation prescriptions in a prescription database, so as to generate a new exclusive electrical stimulation prescription; displaying, by a prescription editing module, the new exclusive electrical stimulation prescription on a doctor's operating terminal for confirmation and/or editing; and receiving, by the prescription editing module, confirmation information of the new exclusive electrical stimulation prescription from the doctor's operating terminal and providing the new exclusive electrical stimulation prescription to the target patient for performing an electrical stimulation treatment.

In at least one embodiment of the present disclosure, the generative artificial intelligence module comprises a large language model (LLM) applying a retrieval-augmented generation (RAG) technique.

In at least one embodiment, the method for generating an electrical stimulation prescription of the present disclosure further comprises storing, based on the confirmation information, the new exclusive electrical stimulation prescription in the prescription database as one of the plurality of electrical stimulation prescriptions, wherein the new exclusive electrical stimulation prescription is associated with one of a plurality of prescription generation rules.

In at least one embodiment, the method for generating an electrical stimulation prescription of the present disclosure further comprises obtaining, by the generative artificial intelligence module, patient information of the target patient, and the generative artificial intelligence module further adjusts the exclusive electrical stimulation prescription based on the patient information, the feedback, and the at least one reference electrical stimulation prescription, so as to generate the new exclusive electrical stimulation prescription.

In at least one embodiment of the present disclosure, the patient information comprises historical data of exclusive electrical stimulation prescriptions previously performed by the target patient.

In at least one embodiment of the present disclosure, the patient information further comprises at least one of basic personal information and medical record information of the target patient.

In at least one embodiment of the present disclosure, the prescription editing module generates a user interface, and the user interface presents at least one carrier signal and a plurality of scripts of the new exclusive electrical stimulation prescription, wherein each of the scripts corresponds to a segment of one of the carrier signals.

In at least one embodiment of the present disclosure, each of the scripts comprises at least one of a duration and an amplitude corresponding to the segment.

In at least one embodiment of the present disclosure, the generative artificial intelligence module performs autonomous learning based on the plurality of electrical stimulation prescriptions to establish a plurality of prescription generation rules, and each of the electrical stimulation prescriptions is associated with one of the plurality of prescription generation rules, wherein the generative artificial intelligence module identifies at least one of the plurality of prescription generation rules conforming to the exclusive electrical stimulation prescription, so as to identify the at least one reference electrical stimulation prescription associated therewith.

In at least one embodiment of the present disclosure, the generative artificial intelligence module generates the exclusive electrical stimulation prescription based on prescription generation information input from the doctor's operating terminal, and, after the prescription editing module receives confirmation information for the exclusive electrical stimulation prescription from the doctor's operating terminal, the generative artificial intelligence module is enabled to obtain the exclusive electrical stimulation prescription.

In at least one embodiment of the present disclosure, the prescription generation information comprises a spoken instruction input via an audio input device of the doctor's operating terminal.

In at least one embodiment, the system for generating an electrical stimulation prescription of the present disclosure comprises a prescription database, a generative artificial intelligence module, and a prescription editing module. The prescription database is configured to store a plurality of electrical stimulation prescriptions; the generative artificial intelligence module is configured to obtain an exclusive electrical stimulation prescription designed for a target patient and feedback from the target patient executing the exclusive electrical stimulation prescription, and to adjust the exclusive electrical stimulation prescription based on the feedback and at least one reference electrical stimulation prescription retrieved from the plurality of electrical stimulation prescriptions, so as to generate a new exclusive electrical stimulation prescription; and the prescription editing module is configured to display the new exclusive electrical stimulation prescription on a doctor's operating terminal for confirmation and/or editing, and after receiving confirmation information of the new exclusive electrical stimulation prescription from the doctor's operating terminal, the prescription editing module is configured to provide the new exclusive electrical stimulation prescription to the target patient for performing an electrical stimulation treatment.

### BRIEF DESCRIPTION OF DRAWINGS

The present disclosure can be more fully understood from the following description of the embodiments, taken in conjunction with the accompanying drawings.

FIG. 1 is a system block diagram of the system for generating an electrical stimulation prescription of the present disclosure.

FIG. 2 is a flowchart of the method for generating an electrical stimulation prescription of the present disclosure.

FIG. 3 is a system block diagram of another embodiment of the system for generating an electrical stimulation prescription of the present disclosure.

FIG. 4 is a flowchart of another embodiment of the method for generating an electrical stimulation prescription of the present disclosure.

FIG. 5 is a schematic diagram of the present disclosure when a doctor edits an exclusive electrical stimulation prescription through a user interface displayed on a doctor's operating terminal.

### DETAILED DESCRIPTION

Since the various aspects and embodiments are merely exemplary and non-limiting, upon reading this specification, a person having ordinary skill in the art will understand that other aspects and embodiments may also be possible without departing from the scope of the present disclosure. The features and advantages of these embodiments will become more apparent from the following detailed description and claims.

As used herein, the terms “a” or “an” are used to describe elements and components described herein. This is solely for convenience of description and imparts a generic sense to the scope of the present disclosure. Accordingly, unless the context clearly indicates otherwise, such terminology is to be understood to mean “one or at least one,” and the singular likewise includes the plural.

As used herein, the terms “include,” “have,” “comprise,” and any similar terms are intended to encompass non-exclusive inclusions. For example, a component or structure having a plurality of elements is not limited to only the elements listed herein; it may include other elements not expressly listed but ordinarily inherent to such component or structure.

The system for generating an electrical stimulation prescription of the present disclosure primarily incorporates generative artificial intelligence (GAI) technology to assist a doctor in issuing or adjusting an exclusive electrical stimulation prescription for a target patient.

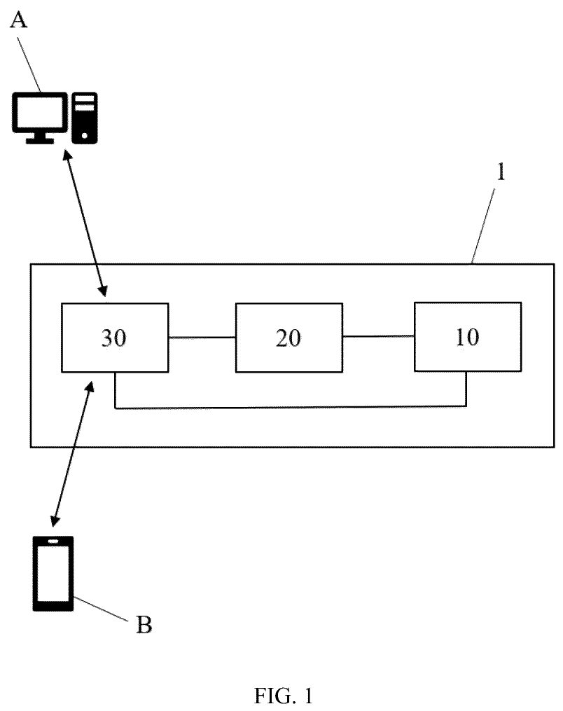

Please refer to FIG. 1, which is a system block diagram of the system for generating an electrical stimulation prescription 1 of the present disclosure. As shown in FIG. 1, the system for generating an electrical stimulation prescription 1 comprises a prescription database 10, a generative artificial intelligence module 20, and a prescription editing module 30, and the prescription database 10, the generative artificial intelligence module 20, and the prescription editing module 30 are electrically connected to each other.

In at least one embodiment of the present disclosure, the prescription database 10 is configured to store a plurality of electrical stimulation prescriptions and a plurality of prescription generation rules that serve as basic reference data for the system for generating an electrical stimulation prescription 1 of the present disclosure. In some embodiments of the present disclosure, each electrical stimulation prescription is associated with at least one of the plurality of prescription generation rules according to the set conditions (such as the patient's condition) corresponding to the time of issuing the each electrical stimulation prescription. In some embodiments of the present disclosure, the prescription database 10 may be implemented as a hardware device having a data storage function, such as a hard disk, but the present disclosure is not limited thereto. For example, the prescription database 10 may also be a combination of a hardware device and related data storage or management software.

In at least one embodiment of the present disclosure, an electrical stimulation prescription is defined as providing at least one carrier signal for an electrical stimulation device to perform an electrical stimulation treatment. By the electrical stimulation device (for example, a general electrotherapy device having a set of adhesive patches) operating according to the carrier signal, corresponding electrical stimulation can be provided to the patient's body parts to achieve a therapeutic effect. In some embodiments of the present disclosure, each carrier signal may provide a single carrier or a combination of a plurality of carriers, and the combination of the plurality of carriers may encompass a plurality of carriers that are continuous or discontinuous and have different durations, waveforms, intensities (amplitudes), or frequencies. In some embodiments of the present disclosure, each electrical stimulation prescription may be an electrical stimulation prescription pre-stored in the system, or may be a newly added electrical stimulation prescription after being stored by the prescription editing module 30 based on a previously adjusted electrical stimulation prescription, wherein each electrical stimulation prescription is an exclusive electrical stimulation prescription issued by a doctor for a specific target patient, and each electrical stimulation prescription has been certified by the doctor and is available for the patient to perform the corresponding electrical stimulation treatment.

In at least one embodiment of the present disclosure, prescription generation rules are defined as rules that, in response to specific set conditions, are associated with corresponding electrical stimulation prescriptions. In some embodiments of the present disclosure, the foregoing set conditions may include basic personal information, physiological status data (for example, range values or threshold values of physiological characteristic parameters), and/or medical record information of a patient for whom an electrical stimulation prescription has been issued, wherein each prescription generation rule may be associated with one or more electrical stimulation prescriptions.

In at least one embodiment of the present disclosure, the generative artificial intelligence module 20 primarily assists a doctor in issuing or adjusting an exclusive electrical stimulation prescription for a target patient by communicating directly with the doctor or by obtaining feedback from the doctor or the target patient. In some embodiments of the present disclosure, the generative artificial intelligence module 20 may be a generative artificial intelligence system trained on textual language. For example, the generative artificial intelligence module 20 comprises a large language model (LLM) applying a retrieval-augmented generation (RAG) technique, such as LLaMA or GPT-4, so that the generative artificial intelligence module 20 may, via the RAG technique, refer to relevant databases to perform language processing or transformation tasks assigned by the system for generating an electrical stimulation prescription 1 of the present disclosure. In some embodiments of the present disclosure, the generative artificial intelligence module 20 may further be used in conjunction with an agent to, on one hand, communicate with the doctor or the target patient in speech or text via the generative artificial intelligence, and, on the other hand, assist in performing related system operations and determinations.

In some embodiments of the present disclosure, the generative artificial intelligence module 20 performs autonomous learning based on a plurality of electrical stimulation prescriptions stored in the prescription database 10 to establish a plurality of prescription generation rules and store the plurality of prescription generation rules in the prescription database 10. In some embodiments of the present disclosure, the generative artificial intelligence module 20 may perform data consolidation and learning according to the set conditions applicable to each electrical stimulation prescription, thereby generalizing and establishing the plurality of prescription generation rules, wherein each electrical stimulation prescription is associated with one of the plurality of prescription generation rules. In some embodiments of the present disclosure, the generative artificial intelligence module 20 may then, according to the plurality of prescription generation rules, identify at least one electrical stimulation prescription as a reference electrical stimulation prescription.

In at least one embodiment of the present disclosure, the generative artificial intelligence module 20 may obtain an exclusive electrical stimulation prescription designed for a target patient, so that the exclusive electrical stimulation prescription may subsequently be adjusted. In some embodiments of the present disclosure, the generative artificial intelligence module 20 may directly obtain an exclusive electrical stimulation prescription that a doctor has issued for the target patient; alternatively, in some embodiments of the present disclosure, the generative artificial intelligence module 20 may, by communicating with the doctor (for example, receiving a spoken instruction from the doctor), generate and obtain an exclusive electrical stimulation prescription designed for the target patient.

In at least one embodiment of the present disclosure, the generative artificial intelligence module 20 may obtain feedback from the target patient executing the exclusive electrical stimulation prescription, so as to serve as reference information for subsequently adjusting the exclusive electrical stimulation prescription. In some embodiments of the present disclosure, the generative artificial intelligence module 20 may directly obtain, from a patient's operating terminal B (for example, an electrotherapy device, a computer, a tablet, or a mobile phone), feedback from the target patient executing the exclusive electrical stimulation prescription (for example, physiological sensing signals detected and returned by sensing elements of the electrotherapy device during the treatment process of the target patient's execution of the exclusive electrical stimulation prescription, or feedback comments returned by the target patient). Alternatively, in some embodiments of the present disclosure, the generative artificial intelligence module 20 may obtain feedback from the target patient executing the exclusive electrical stimulation prescription by communicating with the doctor (for example, receiving the doctor's relayed statements of the target patient's feedback comments or a comprehensive determination made based on such feedback comments).

In at least one embodiment of the present disclosure, the generative artificial intelligence module 20 adjusts an exclusive electrical stimulation prescription based on the feedback and the at least one reference electrical stimulation prescription retrieved from the plurality of electrical stimulation prescriptions, so as to generate a new exclusive electrical stimulation prescription. To adjust the current exclusive electrical stimulation prescription, in addition to the feedback previously received, the generative artificial intelligence module 20 may further identify at least one of a plurality of prescription generation rules that conform to the exclusive electrical stimulation prescription, so as to identify at least one reference electrical stimulation prescription associated therewith from the plurality of electrical stimulation prescriptions, thereby providing additional reference information for subsequently adjusting the exclusive electrical stimulation prescription. In other words, the set conditions applicable to any identified reference electrical stimulation prescription should conform to or be similar to the set conditions applicable to the exclusive electrical stimulation prescription, and, after the exclusive electrical stimulation prescription is adjusted by the generative artificial intelligence module 20 based on the feedback and the at least one reference electrical stimulation prescription, a new exclusive electrical stimulation prescription is generated.

In at least one embodiment of the present disclosure, the prescription editing module 30 displays the new exclusive electrical stimulation prescription on a doctor's operating terminal A (for example, an operating device such as a computer, a tablet, or a mobile phone) for confirmation and/or editing by a doctor at the doctor's operating terminal A. In some embodiments of the present disclosure, the prescription editing module 30 may be a combination of a hardware device having a prescription editing function, such as a processor, and related prescription editing software, and the foregoing prescription editing software may be stored in a hardware device having a data storage function, but the present disclosure is not limited thereto. For example, the prescription editing module 30 may also be standalone software or hardware. In some embodiments of the present disclosure, the prescription editing module 30 may generate a user interface, and the doctor's operating terminal A may, by executing a related application, display the user interface to present at least one carrier signal and a plurality of scripts of the new exclusive electrical stimulation prescription, wherein each of the scripts corresponds to a segment of any one of the carrier signals. In some embodiments of the present disclosure, each of the scripts comprises at least one of a duration and an amplitude corresponding to the segment.

In at least one embodiment of the present disclosure, a doctor may, via the user interface displayed on the doctor's operating terminal A, edit and/or modify any one of the scripts of the new exclusive electrical stimulation prescription, and determine whether the content of the editing and/or modification is correct based on the segment of the carrier signal correspondingly presented according to the modified script. In some embodiments of the present disclosure, upon the doctor's confirmation, the doctor may transmit, via the user interface of the doctor's operating terminal A, confirmation information for the modified new exclusive electrical stimulation prescription to the prescription editing module 30. In some embodiments of the present disclosure, after receiving the confirmation information, the prescription editing module 30 may store the new exclusive electrical stimulation prescription in the prescription database 10 and associate it with one of a plurality of prescription generation rules, thereby adding the new exclusive electrical stimulation prescription as one of the plurality of electrical stimulation prescriptions.

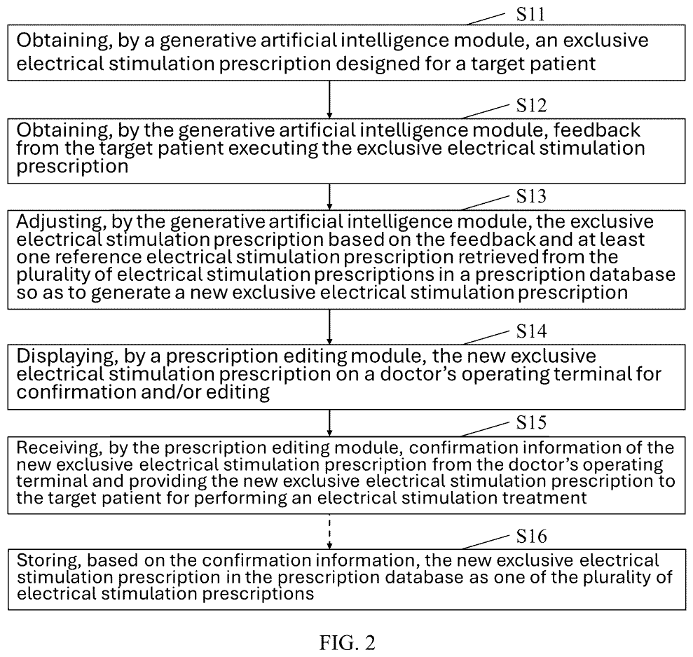

Please refer to FIG. 1 and FIG. 2 together, wherein FIG. 2 is a flowchart of the method for generating an electrical stimulation prescription of the present disclosure. As shown in FIG. 1 and FIG. 2, in at least one embodiment of the present disclosure, the method for generating an electrical stimulation prescription is executed by the aforementioned system for generating an electrical stimulation prescription 1, and the method for generating an electrical stimulation prescription comprises the following steps:
Step S11: obtaining, by the generative artificial intelligence module 20, an exclusive electrical stimulation prescription designed for a target patient.

Specifically, the method for generating an electrical stimulation prescription of the present disclosure obtains, by the generative artificial intelligence module 20, an exclusive electrical stimulation prescription designed for a target patient, so that the exclusive electrical stimulation prescription may subsequently be adjusted. As described above, in some embodiments of the present disclosure, the generative artificial intelligence module 20 may directly obtain an exclusive electrical stimulation prescription that the doctor has issued for the target patient. For example, the generative artificial intelligence module 20 may obtain the exclusive electrical stimulation prescription, as previously issued and stored by the doctor, through various hardware storage devices (for example, a hard disk, a flash drive, or a memory card) or through network storage media (for example, a cloud platform or a server).

In some embodiments of the present disclosure, the generative artificial intelligence module 20 may also generate and obtain an exclusive electrical stimulation prescription designed for a target patient by communicating directly with the doctor. For example, the doctor may input, via the prescription editing module 30, prescription generation information on the user interface displayed on the doctor's operating terminal A (for example, a text instruction input by the doctor through input devices such as a keyboard or a handwriting pad of the doctor's operating terminal A, or a spoken instruction input via an audio input device such as a microphone). After receiving the prescription generation information, the prescription editing module 30 may transmit the prescription generation information to the generative artificial intelligence module 20, and the generative artificial intelligence module 20 may generate, based on the prescription generation information, a plurality of initial scripts for the target patient's exclusive electrical stimulation prescription and present, via the prescription editing module 30, this exclusive electrical stimulation prescription on the user interface displayed on the doctor's operating terminal A, wherein the exclusive electrical stimulation prescription presented on the user interface includes the plurality of initial scripts and carrier signals correspondingly presented after conversion according to those initial scripts, so that the doctor may directly confirm the content of the exclusive electrical stimulation prescription via the user interface and adjust or modify the carrier signals. When the doctor confirms that the exclusive electrical stimulation prescription is correct, the doctor may input and transmit, via the user interface of the doctor's operating terminal A, confirmation information for the exclusive electrical stimulation prescription, and, after receiving the confirmation information transmitted from the doctor's operating terminal A, the generative artificial intelligence module 20 may obtain the exclusive electrical stimulation prescription.
Step S12: obtaining, by the generative artificial intelligence module 20, feedback from the target patient executing the exclusive electrical stimulation prescription.

Following Step S11, after obtaining an exclusive electrical stimulation prescription designed for the target patient, the method for generating an electrical stimulation prescription of the present disclosure may then obtain, by the generative artificial intelligence module 20, feedback from the target patient executing the exclusive electrical stimulation prescription, to serve as reference information for subsequently adjusting the exclusive electrical stimulation prescription. As described above, in some embodiments of the present disclosure, the feedback may be provided by the target patient via the patient's operating terminal B, or may be provided via the doctor's operating terminal A as the doctor's feedback comments on the target patient or as the doctor's comprehensive determination made based on such feedback comments.
Step S13: adjusting, by the generative artificial intelligence module 20, the exclusive electrical stimulation prescription based on the feedback and the at least one reference electrical stimulation prescription retrieved from the plurality of electrical stimulation prescriptions in the prescription database 10, so as to generate a new exclusive electrical stimulation prescription.

After obtaining, in Step S12, the feedback from the target patient executing the exclusive electrical stimulation prescription, the method for generating an electrical stimulation prescription of the present disclosure may then identify, by the generative artificial intelligence module 20, at least one reference electrical stimulation prescription from the plurality of electrical stimulation prescriptions in the prescription database 10, and, based on the feedback previously obtained and the at least one reference electrical stimulation prescription, adjust the exclusive electrical stimulation prescription, so as to generate an adjusted new exclusive electrical stimulation prescription. As described above, in some embodiments of the present disclosure, the generative artificial intelligence module 20 may compare the set conditions applicable to the current exclusive electrical stimulation prescription with the plurality of prescription generation rules in the prescription database 10 to identify at least one prescription generation rule that more closely conforms to or approximates the set conditions applicable to the exclusive electrical stimulation prescription. Then, the generative artificial intelligence module 20 may, according to the at least one more closely compliant or approximating prescription generation rule, identify from the plurality of electrical stimulation prescriptions the at least one reference electrical stimulation prescription associated therewith, thereby providing additional reference information for adjusting the exclusive electrical stimulation prescription. Thereafter, the generative artificial intelligence module 20 may adjust the exclusive electrical stimulation prescription based on the feedback from the target patient executing the exclusive electrical stimulation prescription and the at least one reference electrical stimulation prescription associated with the exclusive electrical stimulation prescription, so as to generate the adjusted new exclusive electrical stimulation prescription.
Step S14: displaying, by the prescription editing module 30, the new exclusive electrical stimulation prescription on a doctor's operating terminal A for confirmation and/or editing.

After Step S13 for adjusting the exclusive electrical stimulation prescription and generating a new exclusive electrical stimulation prescription, the method for generating an electrical stimulation prescription of the present disclosure may present, by the prescription editing module 30, the new exclusive electrical stimulation prescription on a doctor's operating terminal A for the doctor to confirm and/or edit the content of the new exclusive electrical stimulation prescription. As described above, in some embodiments of the present disclosure, the prescription editing module 30 may present, on the user interface displayed on the doctor's operating terminal A, at least one carrier signal and a plurality of scripts of the new exclusive electrical stimulation prescription, so that the doctor may, via the user interface, edit and/or modify any one of the scripts of the new exclusive electrical stimulation prescription, and the modified script may present, on the user interface, a segment of the carrier signal corresponding to the modified content, for the doctor to determine whether the content of the editing and/or modification is correct.
Step S15: receiving, by the prescription editing module 30, confirmation information of the new exclusive electrical stimulation prescription from the doctor's operating terminal A, and providing the new exclusive electrical stimulation prescription to the target patient for performing an electrical stimulation treatment.

After Step S14 for presenting the new exclusive electrical stimulation prescription on the doctor's operating terminal A for confirmation and/or editing, the doctor may transmit, via the user interface displayed on the doctor's operating terminal A, confirmation information for the new exclusive electrical stimulation prescription to the prescription editing module 30. Thereafter, upon receiving the confirmation information, the prescription editing module 30 may determine that the new exclusive electrical stimulation prescription has been certified by the doctor, and then provide the new exclusive electrical stimulation prescription to the patient's operating terminal B as the latest prescription for the target patient to perform an electrical stimulation treatment, replacing the original exclusive electrical stimulation prescription prior to the adjustment. In some embodiments of the present disclosure, because the new exclusive electrical stimulation prescription is generated by the generative artificial intelligence module 20 based on the feedback and the reference electrical stimulation prescriptions applicable to similar set conditions, and by adjusting the original exclusive electrical stimulation prescription, the new exclusive electrical stimulation prescription can better meet the target patient's current needs and status, thereby achieving an improved effect on electrical stimulation treatment.

In at least one embodiment of the present disclosure, the method for generating an electrical stimulation prescription may further include Step S16 for storing, based on the confirmation information, the new exclusive electrical stimulation prescription in the prescription database 10 as one of the plurality of electrical stimulation prescriptions, wherein the new exclusive electrical stimulation prescription is associated with one of a plurality of prescription generation rules.

After Step S15 for receiving the confirmation information for the new exclusive electrical stimulation prescription, the method for generating an electrical stimulation prescription of the present disclosure may further store, by the prescription editing module 30, based on the confirmation information, the new exclusive electrical stimulation prescription in the prescription database 10 as one of the plurality of electrical stimulation prescriptions. As described above, in some embodiments of the present disclosure, after receiving the confirmation information, the prescription editing module 30 may determine that the new exclusive electrical stimulation prescription is indeed an electrical stimulation prescription certified by the doctor, and therefore store the new exclusive electrical stimulation prescription in the prescription database 10, so that the new exclusive electrical stimulation prescription is further added as one of the plurality of electrical stimulation prescriptions. In some embodiments of the present disclosure, the prescription editing module 30 may associate, according to the set conditions applicable to the new exclusive electrical stimulation prescription, the new exclusive electrical stimulation prescription with one of a plurality of prescription generation rules. Accordingly, the new exclusive electrical stimulation prescription may subsequently serve as a reference electrical stimulation prescription, thereby increasing the number of electrical stimulation prescriptions in the prescription database 10 that can be referenced and applied.

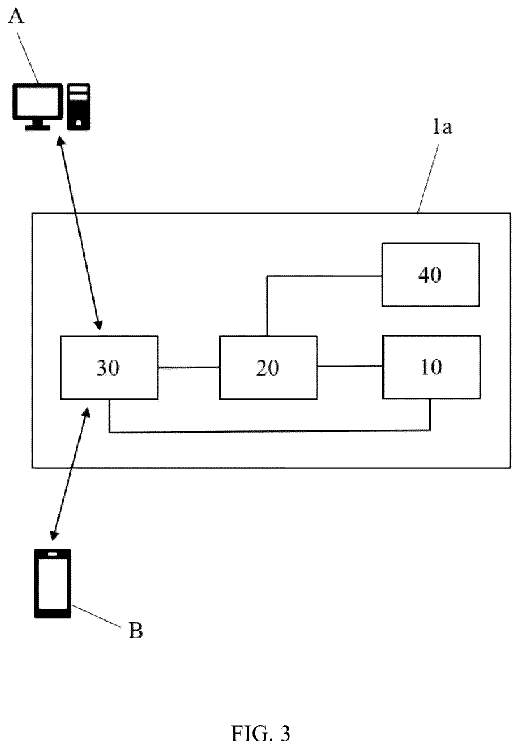

Please refer to FIG. 3, which is a system block diagram of another embodiment of the system for generating an electrical stimulation prescription 1a of the present disclosure. As shown in FIG. 3, in at least one embodiment of the present disclosure, the system for generating an electrical stimulation prescription 1a further includes a patient database 40, which is electrically connected to the generative artificial intelligence module 20. In some embodiments of the present disclosure, the patient database 40 is mainly used to store patient information as reference data for the generative artificial intelligence module 20 to adjust the target patient's exclusive electrical stimulation prescription. In some embodiments of the present disclosure, the patient database 40 may be configured as a hardware device having a data storage function, such as a hard disk, but the present disclosure is not limited thereto. For example, the patient database 40 may also be a combination of a hardware device and related data storage or management software.

In some embodiments of the present disclosure, patient information may include historical data of exclusive electrical stimulation prescriptions previously performed by the target patient. In other words, by means of the historical data, each exclusive electrical stimulation prescription previously performed by the target patient and the related content thereof can be known, so as to provide the generative artificial intelligence module 20 with data to learn from or to reference. In at least one embodiment of the present disclosure, the patient information may further include basic personal information and medical record information of the target patient. In other words, by means of the basic personal information and medical record information together with the foregoing historical data, it can be known under what physiological conditions the target patient was issued the corresponding exclusive electrical stimulation prescriptions, so as to provide the generative artificial intelligence module 20 with data to learn from or to reference.

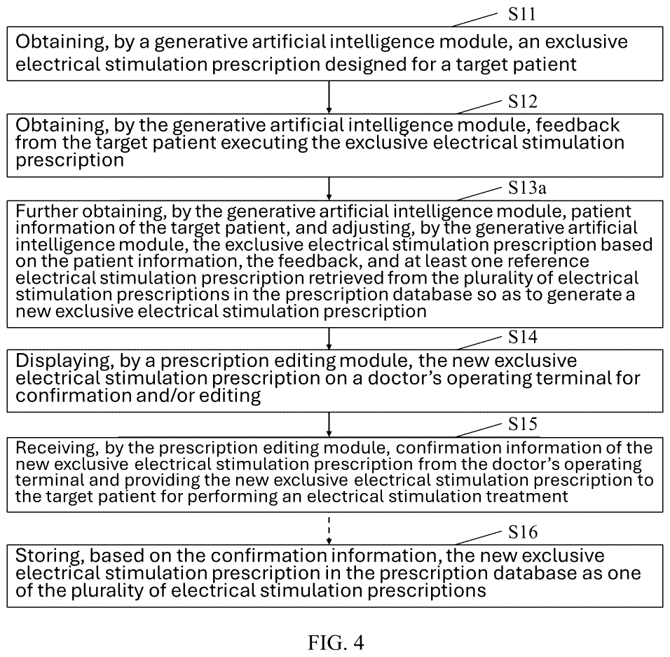

Please refer to FIG. 3 and FIG. 4 together, wherein FIG. 4 is a flowchart of another embodiment of the method for generating an electrical stimulation prescription of the present disclosure. As shown in FIG. 3 and FIG. 4, in at least one embodiment of the present disclosure, the method for generating an electrical stimulation prescription is executed by the aforementioned system for generating an electrical stimulation prescription 1a, and the method for generating an electrical stimulation prescription replaces Step S13 of FIG. 2 with Step S13a.
Step S13a: further obtaining, by the generative artificial intelligence module 20, patient information of the target patient, and adjusting, by the generative artificial intelligence module 20, the exclusive electrical stimulation prescription based on the patient information, the feedback, and the at least one reference electrical stimulation prescription retrieved from the plurality of electrical stimulation prescriptions in the prescription database 10, so as to generate a new exclusive electrical stimulation prescription.

After Step S12 for obtaining feedback from the target patient executing the exclusive electrical stimulation prescription, the method for generating an electrical stimulation prescription of the present disclosure may further obtain, by the generative artificial intelligence module 20, patient information of the target patient from the patient database 40 to serve as additional reference information for adjusting the exclusive electrical stimulation prescription. In at least one embodiment of the present disclosure, with the patient information thus obtained, the generative artificial intelligence module 20 may further learn and understand the set conditions corresponding to the currently issued exclusive electrical stimulation prescription, thereby facilitating the generative artificial intelligence module 20 to identify, from the plurality of electrical stimulation prescriptions in the prescription database 10, at least one reference electrical stimulation prescription with greater accuracy and, based on the patient information obtained, the feedback, and the at least one reference electrical stimulation prescription, to adjust the exclusive electrical stimulation prescription, so as to generate an adjusted new exclusive electrical stimulation prescription.

In at least one embodiment of the present disclosure, the patient's operating terminal B may download and install in advance an application from a cloud platform, and, via the user interface presented upon execution of the application, log in to the cloud platform and use the related user interfaces to download, from the cloud platform, the exclusive electrical stimulation prescription designated for and applicable to the target patient. In some embodiments of the present disclosure, the downloaded exclusive electrical stimulation prescription is stored in the patient's operating terminal B. In addition, the target patient may, via the foregoing user interface, fill out and return a questionnaire to the cloud platform as feedback comments for the doctor's reference or for subsequent adjustment of the electrical stimulation prescription.

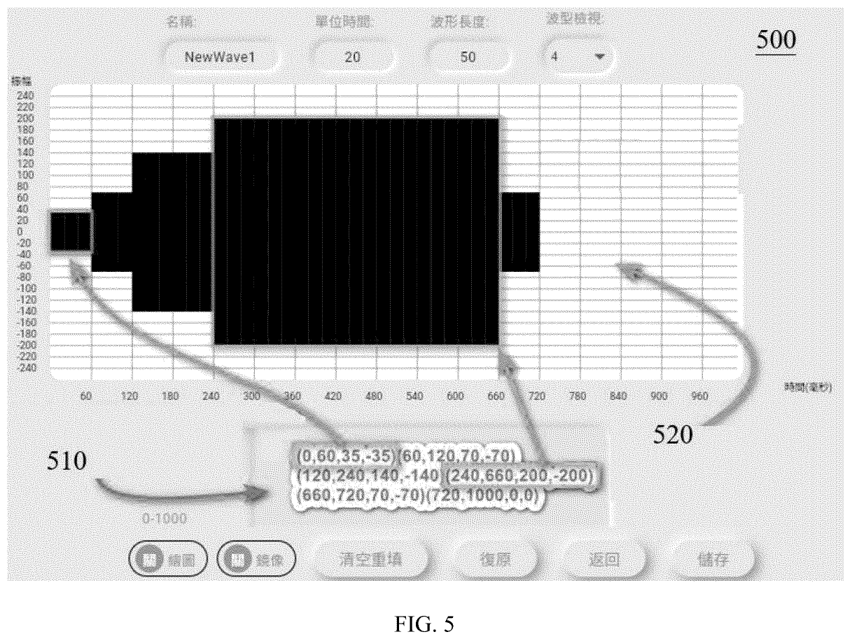

As shown in FIG. 5, a schematic diagram is further provided for illustrating the editing of an exclusive electrical stimulation prescription by a doctor via a user interface 500 displayed on a doctor's operating terminal A in the present disclosure. In some embodiments of the present disclosure, the user interface 500 includes a text script editing area 510 and a carrier signal display area 520, wherein the text script editing area 510 is used to edit scripts for the carrier signal of an electrical stimulation prescription to be generated, and the carrier signal display area 520 is used to display the carrier signal generated from the scripts. In some embodiments of the present disclosure, the scripts may include numerical information such as start time, end time, upper amplitude, and lower amplitude, and the present disclosure is not particularly limited thereto.

For example, as shown in FIG. 5, in some embodiments of the present disclosure, if the edited start time and end time are the 0 millisecond and the 60 millisecond, respectively, and the values of the upper amplitude and lower amplitude are 35 and −35, respectively, then the script may be displayed as “(0,60,35, −35).” Likewise, when the script is displayed as “(240,660,200, −200),” it indicates that the edited start time and end time are the 240 millisecond and the 660 millisecond, respectively, and that the values of the upper amplitude and lower amplitude are 200 and −200, respectively.

In some embodiments of the present disclosure, in addition to editing the desired carrier signal via the text script editing area 510, a doctor may also depict the desired carrier signal in the carrier signal display area 520 by selecting rectangular blocks formed by grid lines therein. In some embodiments of the present disclosure, after the doctor depicts the desired carrier signal by selecting the rectangular blocks in the carrier signal display area 520, the user interface 500 may also present, in the text script editing area 510, the corresponding script of the depicted carrier signal, thereby facilitating the doctor's further confirmation of whether the modified numerical values are correct.

Accordingly, the system for generating an electrical stimulation prescription of the present disclosure can communicate with a doctor by the generative artificial intelligence module or obtain, from the doctor, an exclusive electrical stimulation prescription for a target patient, and, based on the feedback from the target patient executing the exclusive electrical stimulation prescription and as at least one reference electrical stimulation prescription having similar set conditions in the exclusive electrical stimulation prescription and patient information, adjust the exclusive electrical stimulation prescription. A doctor may also utilize the user interface of the system for generating an exclusive electrical stimulation prescription to confirm and/or modify the adjusted new exclusive electrical stimulation prescription, thereby assisting the doctor in issuing or adjusting the exclusive electrical stimulation prescription, reducing the doctor's workload, and enabling the exclusive electrical stimulation prescription to be adjusted to better suit the target patient's current condition.

The foregoing embodiments are mainly for illustrative purposes and are not intended to limit the embodiments of the claimed subject matter or applications thereof. Although at least one exemplary embodiment has been presented in the foregoing description, it should be understood that numerous variations may still be made to the present disclosure. It should likewise be understood that the embodiments described herein are not intended to limit in any way the scope, use, or manner of practicing the claimed subject matter. On the contrary, the foregoing description will provide a convenient guide to a person of ordinary skill in the art for implementing one or more of the embodiments described. Furthermore, various changes may be made to the functions and arrangements of components without departing from the scope defined by the claims, and the claims encompass any known equivalents and all foreseeable equivalents as of the filing of this patent application.

What is claimed is:

1. A method for generating an electrical stimulation prescription, comprising:
obtaining, by a generative artificial intelligence module, an exclusive electrical stimulation prescription designed for a target patient;
obtaining, by the generative artificial intelligence module, feedback from the target patient executing the exclusive electrical stimulation prescription;
adjusting, by the generative artificial intelligence module, the exclusive electrical stimulation prescription based on the feedback and at least one reference electrical stimulation prescription retrieved from a plurality of electrical stimulation prescriptions in a prescription database, so as to generate a new exclusive electrical stimulation prescription;
displaying, by a prescription editing module, the new exclusive electrical stimulation prescription on a doctor's operating terminal for confirmation and/or editing; and
receiving, by the prescription editing module, confirmation information of the new exclusive electrical stimulation prescription from the doctor's operating terminal and providing the new exclusive electrical stimulation prescription to the target patient for performing an electrical stimulation treatment.

2. The method of claim 1, wherein the generative artificial intelligence module comprises a large language model applying a retrieval-augmented generation technique.

3. The method of claim 1, further comprising storing, based on the confirmation information, the new exclusive electrical stimulation prescription in the prescription database as one of the plurality of electrical stimulation prescriptions, wherein the new exclusive electrical stimulation prescription is associated with one of a plurality of prescription generation rules.

4. The method of claim 1, further comprising obtaining, by the generative artificial intelligence module, patient information of the target patient, wherein the generative artificial intelligence module further adjusts the exclusive electrical stimulation prescription based on the patient information, the feedback, and the at least one reference electrical stimulation prescription, so as to generate the new exclusive electrical stimulation prescription.

5. The method of claim 4, wherein the patient information comprises historical data of the exclusive electrical stimulation prescriptions previously performed by the target patient, and wherein the patient information further comprises at least one of basic personal information and medical record information of the target patient.

6. The method of claim 1, wherein the prescription editing module generates a user interface, and the user interface is configured to present at least one carrier signal and a plurality of scripts of the new exclusive electrical stimulation prescription, and wherein each of the scripts corresponds to a segment of one of the carrier signals.

7. The method of claim 6, wherein each of the scripts comprises at least one of a duration and an amplitude corresponding to the segment.

8. The method of claim 1, wherein the generative artificial intelligence module performs autonomous learning based on the plurality of electrical stimulation prescriptions to establish a plurality of prescription generation rules, and each of the electrical stimulation prescriptions is associated with one of the plurality of prescription generation rules, and wherein the generative artificial intelligence module identifies at least one of the plurality of prescription generation rules conforming to the exclusive electrical stimulation prescription, so as to identify the at least one reference electrical stimulation prescription associated therewith.

9. The method of claim 8, wherein the generative artificial intelligence module generates the exclusive electrical stimulation prescription based on prescription generation information input from the doctor's operating terminal, and after the prescription editing module receives the confirmation information for the exclusive electrical stimulation prescription from the doctor's operating terminal, the generative artificial intelligence module is enabled to obtain the exclusive electrical stimulation prescription.

10. The method of claim 9, wherein the prescription generation information comprises a spoken instruction input via an audio input device of the doctor's operating terminal.

11. A system for generating an electrical stimulation prescription, comprising:
a prescription database configured to store a plurality of electrical stimulation prescriptions;
a generative artificial intelligence module configured to obtain an exclusive electrical stimulation prescription designed for a target patient and feedback from the target patient executing the exclusive electrical stimulation prescription, and to adjust the exclusive electrical stimulation prescription based on the feedback and at least one reference electrical stimulation prescription retrieved from the plurality of electrical stimulation prescriptions, so as to generate a new exclusive electrical stimulation prescription; and
a prescription editing module configured to display the new exclusive electrical stimulation prescription on a doctor's operating terminal for confirmation and/or editing, and after receiving confirmation information of the new exclusive electrical stimulation prescription from the doctor's operating terminal, to provide the new exclusive electrical stimulation prescription to the target patient for performing an electrical stimulation treatment.

12. The system of claim 11, wherein the generative artificial intelligence module comprises a large language model applying a retrieval-augmented generation technique.

13. The system of claim 11, wherein the prescription editing module stores, based on the confirmation information, the new exclusive electrical stimulation prescription in the prescription database as one of the plurality of electrical stimulation prescriptions, and wherein the new exclusive electrical stimulation prescription is associated with one of a plurality of prescription generation rules.

14. The system of claim 11, further comprising a patient database, wherein the generative artificial intelligence module is configured to obtain patient information of the target patient from the patient database, and the generative artificial intelligence module is further configured to adjust the exclusive electrical stimulation prescription based on the patient information, the feedback, and the at least one reference electrical stimulation prescription, so as to generate the new exclusive electrical stimulation prescription.

15. The system of claim 14, wherein the patient information comprises historical data of exclusive electrical stimulation prescriptions previously performed by the target patient, and wherein the patient information further comprises at least one of basic personal information and medical record information of the target patient.

16. The system of claim 11, wherein the prescription editing module is configured to generate a user interface, and the user interface is configured to present at least one carrier signal and a plurality of scripts of the new exclusive electrical stimulation prescription, and wherein each of the scripts corresponds to a segment of one of the carrier signals.

17. The system of claim 16, wherein each of the scripts comprises at least one of a duration and an amplitude corresponding to the segment.

18. The system of claim 11, wherein the generative artificial intelligence module is configured to perform autonomous learning based on the plurality of electrical stimulation prescriptions to establish a plurality of prescription generation rules and store the plurality of prescription generation rules in the prescription database, and each of the electrical stimulation prescriptions is associated with one of the plurality of prescription generation rules, and wherein the generative artificial intelligence module is configured to identify at least one of the plurality of prescription generation rules conforming to the exclusive electrical stimulation prescription, so as to identify the at least one reference electrical stimulation prescription associated therewith.

19. The system of claim 11, wherein the generative artificial intelligence module is configured to generate the exclusive electrical stimulation prescription based on prescription generation information input from the doctor's operating terminal, and after the prescription editing module receives confirmation information for the exclusive electrical stimulation prescription from the doctor's operating terminal, the generative artificial intelligence module is enabled to obtain the exclusive electrical stimulation prescription.

20. The system of claim 19, wherein the prescription generation information comprises a spoken instruction input via an audio input device of the doctor's operating terminal.
